(19) United States
(12) Plant Patent Application Publication
(10) Pub. No.: US 2016/0242340 P1
(43) Pub. Date: Aug. 18, 2016

(54) Grapevine named 'FLEURTAI'

(50) ;

(71) Applicants: Università degli Studi di Udine, Udine UD (IT); Istituto di Genomica Applicata, Udine UD (IT)

(72) Inventors: Simone Diego CASTELLARIN, Vancouver (CA); Guido CIPRIANI, Faedis (IT); Gabriele DI GASPERO, Cividale del Friuli UD (IT); Michele MORGANTE, Tricesimo UD (IT); Enrico PETERLUNGER, Codroipo UD (IT); Raffaele TESTOLIN, Udine (IT)

(73) Assignees: Università degli Studi di Udine, Udine UD (IT); Istituto di Genomica Applicata, Udine UD (IT)

(21) Appl. No.: 14/545,111

(22) Filed: Mar. 27, 2015

(30) Foreign Application Priority Data
Feb. 13, 2015 (QZ) 2015/0363

(51) Int. Cl. A01H 5/00

(52) U.S. Cl. PLT/207

(57) ABSTRACT

A new and distinct variety of grapevine plant, named ‘FLEURTAI’, primarily adapted to the growing conditions of the temperate regions and characterized by its strong vigor; erect growth habit; small to medium circular shaped leaves with medium texture; medium sized berry clusters with golden-green berries that have an amber nuance on the sunny side of the berry, soft flesh, neutral taste, and no flesh coloration primarily used for wine; early harvesting time; and resistance to winter temperatures (to −23° C.), resistance to downy mildew, and tolerance to powdery mildew.

### LATIN NAME OF THE GENUS AND SPECIES OF THE PLANT CLAIMED

Vitis×vinifera (hybrid)

### VARIETY NAME

‘FLEURTAI’

### BACKGROUND OF THE INVENTION

The present invention relates to a new and distinct summer/fall bearing grapevine variety, botanically known as Vitis vinifera, and hereinafter referred to by the name ‘FLEURTAI’.

The new grapevine ‘FLEURTAI’ is a product of a controlled breeding program conducted by the inventors in Udine, Italy. The objective of the breeding program was to develop a new grapevine variety particularly characterized by resistance to cold (<−20° C.), resistance to downy mildew (Plasmopara viticola), and tolerance to powdery mildew (Uncinula necator).

The new grapevine ‘FLEURTAI’ originated from a cross made by the inventors in Udine, Italy. The female or seed parent is the grapevine variety, Vitis vinifera cv. ‘Tocai Friulano’ (unpatented), and the male or pollen parent is the grapevine variety, Vitis cv. ‘20/3’ (Bianca×SK77-4/5) (unpatented).

The new grapevine ‘FLEURTAI’ was discovered and selected by the inventors as a single flowering plant within the progeny of the stated cross in a controlled environment in 2002 in Udine, Italy. Asexual reproduction of the new grapevine variety by grafting was first performed in February 2004 in Rauscedo, Friuli Venezia Giulia region, Italy, and has demonstrated that the combination of characteristics as herein disclosed for the new cultivar are firmly fixed and retained through successive generations of asexual reproduction. The new cultivar reproduces true to type.

### SUMMARY OF THE INVENTION

‘FLEURTAI’ is primarily adapted to the climate and growing conditions of the temperate regions with average yearly temperature about 13° C., minimum winter temperature about −20° C., annual rainfall around 700-1500 mm of rain (e.g. North-Eastern Italy, Friuli). This region provides the necessary year-round temperatures required for it to produce and maintain a strong vigorous plant with consistent fruit production from April through November on primocanes and in the ensuing year from April through November on the floricanes.

The following traits have been repeatedly observed and are determined to be unique characteristics of ‘FLEURTAI’, which in combination distinguish this grapevine plant as a new and distinct variety.

1. Strong vigor;

2. Erect growth habit;

3. Small to medium circular shaped leaves with medium to dark green upper surfaces and light green lower surfaces and medium texture;

4. Medium size, loose to medium dense, conical berry clusters with two middle size wings; golden-green color berries with amber nuances on the sunny side of the berry, soft flesh, neutral taste, no flesh coloration; primarily for wine; maturing in late August (Middle Friuli, North-Eastern Italy);

5. Primocanes and floricanes with oblate cross section, brownish color, no lenticels, and short-middle internodes (diameter: 8 mm);

6. Late August harvesting time (Middle Friuli, North-Eastern Italy); and

7. Resistance to winter temperature (−23° C.), resistance to downy mildew, and tolerance to powdery mildew.

Plants of the new grapevine ‘FLEURTAI’ differ from plants of the parents, Vitis vinifera ‘Tocai Friulano’ (unpatented) and Vitis cross ‘20/3’ (unpatented), in the following characteristics described in Table 1.

TABLE 1
Comparison with Parent Varieties

| | | Female Parent | Male Parent |
|---|---|---|---|
| Charac- | New Cultivar | ‘Tocai Friulano’ | ‘20/3’ |
| teristic | ‘FLEURTAI’ | (unpatented) | (unpatented) |
| vigor | strong | strong | medium |
| growth | erect | semi-erect | semi-erect |
| habit | | | |
| leaf | small to medium | medium size, | Medium size, |
| | size, medium to | light green color | light green color |
| | dark green color | (upper surface), | (upper surface), |
| | (upper surface), | green color (low- | green color (lower |
| | light green color | er surface), very | surface), no hairs |
| | (lower surface), | few hairs in both | in both surfaces, |
| | no hairs in both | surfaces, circular | wedge-shaped- |
| | surfaces, circular | shape, medium | kidney-shaped, |
| | shape, medium | texture | medium texture |
| | texture | | |
| cluster | very low-low | low weight, con- | low weight, coni- |
| | weight, conical | ical, with 1-2 | cal, with 1-2 wings, |
| | with two middle | wings, dense, ber- | medium dense, |
| | size wings, loose | ry skin with green- | berry skin with |
| | or medium dense, | yellow color, soft | green-yellow color, |
| | berry skin with | flesh, neutral taste, | slightly firm flesh, |
| | golden-green color | no flesh coloration, | neutral taste, no |
| | with amber nuances | use: wine, maturity: | flesh coloration, |
| | in the sunny side of | medium (early Sep- | use: wine, maturity: |
| | berry, soft flesh, | tember at Udine, | early (late August, |
| | neutral taste, no | North-Eastern | Middle Friuli, |
| | flesh coloration, | Italy) | North-Eastern |
| | use: wine, maturity: | | Italy) |
| | early (late August, | | |
| | Middle Friuli, | | |
| | North-Eastern | | |
| | Italy) | | |
| primo- | oblate cross section, | circular cross sec- | oblate cross section, |
| cane and | brownish color, | tion, brownish col- | brownish color, |
| floricane | lenticels: absent, | or, lenticels: absent, | lenticels: absent, |
| | short-medium | short-medium inter- | medium internodes, |
| | internodes, diam- | nodes, diameter | diameter small |
| | eter: small | small (about | (about 8 mm) |
| | (about 8 mm) | 8 mm) | |
| harvest- | early (last decade | Early-medium (ear- | Early (end August, |
| ing time | August, Middle | ly September, Mid- | Middle Friuli, |
| | Friuli, North- | dle Friuli, North- | North-Eastern |
| | Eastern Italy) | Eastern Italy) | Italy) |
| resis- | resistant to winter | Average resistance | resistant to winter |
| tances | temperature | to winter tempera- | temperature not |
| | (−23° C.), | ture (−15° C.), sus- | checked, resistant |
| | resistant to downy | ceptible to downy | to downy mildew, |
| | mildew, tolerant to | mildew, susceptible | resistant to pow- |
| | powdery mildew | to powdery mildew | dery mildew |

Of the many commercial cultivars known to the present inventor, the most similar to the new grapevine ‘FLEURTAI’ is the female parent ‘Tocai Friulano’, to which a comparison has been provided above.

### BRIEF DESCRIPTION OF THE PHOTOGRAPHS

The accompanying photographs illustrate the overall appearance of the new grapevine ‘FLEURTAI’ showing the colors as true as is reasonably possible with colored reproductions of this type. Colors in the photographs may differ slightly from the color values cited in the detailed botanical description, which accurately describe the color of ‘FLEURTAI’.

FIG. 1A and FIG. 1B show a typical fruit cluster of ‘FLEURTAI’ taken on Aug. 25, 2012, in Udine, Italy.

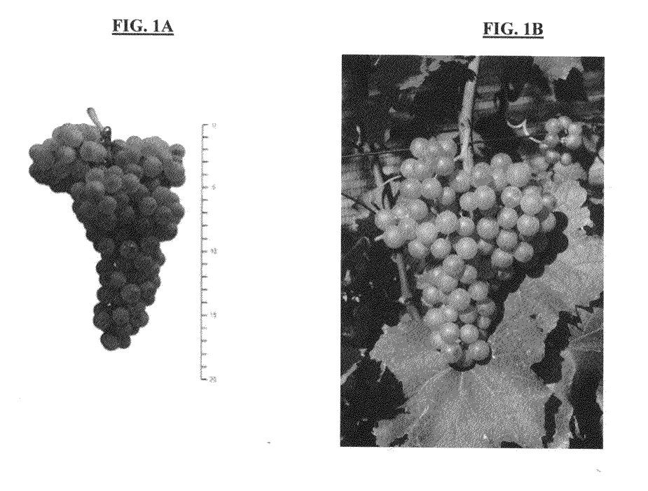

FIG. 2 shows a typical mature leaf (upper surface, left and lower surface, right) of ‘FLEURTAI’ taken on Jun. 15, 2012, in Udine, Italy.

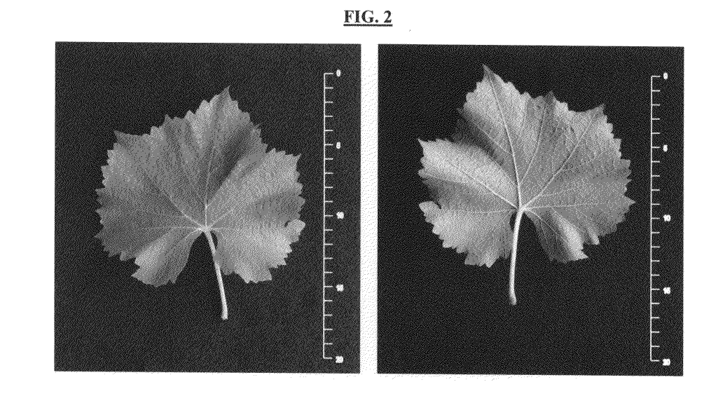

FIG. 3 shows a typical mature vine of ‘FLEURTAI’ taken on Aug. 3, 2012.

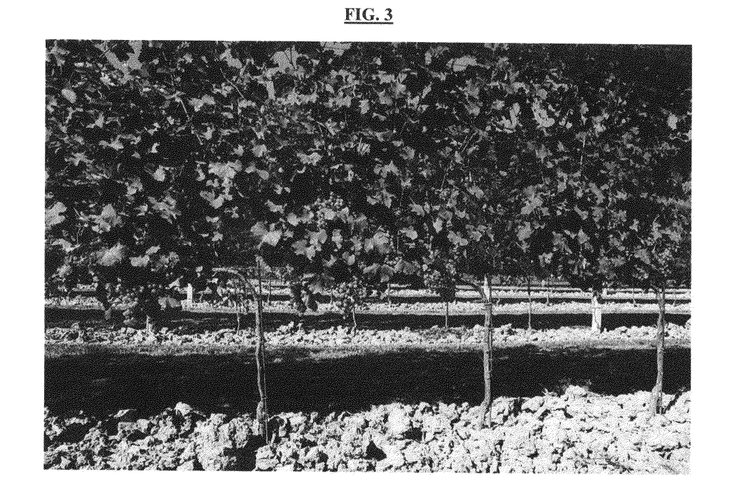

FIG. 4 shows a close-up view of typical mature fruit of ‘FLEURTAI’ taken on Aug. 25, 2012, in Udine, Italy

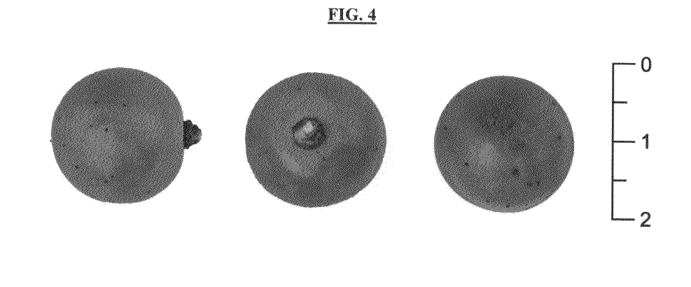

### DETAILED BOTANICAL DESCRIPTION

The following description of ‘FLEURTAI’ unless otherwise noted, is based on observations taken during the 2011, 2012, and 2013 growing seasons(s) in Udine, Italy and Fossalon di Grado (GO), Italy, from plants dug from a nursery located in Vivai Cooperativi Rauscedo, Rauscedo (PN), Italy during the beginning of December 2007 and planted approximately 16 to 20 weeks later in Udine, Italy and Fossalon di Grado (GO), Italy. The phenotypical descriptions and color designations stated for the new variety may vary, depending upon variations in environmental factors, including weather (temperature, humidity and light intensity), day length, soil type, location and cultural conditions. ‘FLEURTAI’ has not been observed under all possible environmental conditions.

| | | |
|---|---|---|
| | | ‘TOCAI |
| | | FRIULANO’ |
| Characteristic | ‘FLEURTAI’ | (unpatented) |

GENERAL

| | | |
|---|---|---|
| Resistance to | Resistant to downy | Susceptible to downy |
| pest/disease | mildew, tolerant to | mildew and to pow- |
| | powdery mildew | dery mildew |
| Resistance to | Resistant to winter | Resistant to winter |
| natural elements | cold down to −23° C. | cold down to −15° C. |

VINE

| | | |
|---|---|---|
| Vigor | strong | strong |
| Trunk diameter | 6 cm (6 yrs.-old plant) | 6.5 |
| Bark | | |
| color | Dark brown | Dark brown |
| underbark color | Light brown | Light brown |
| texture | n.a. | n.a. |
| Canes | | |
| length | Internode length | Internode length |
| | cm 10.5 | about cm 10 |
| width | 8 mm | 8 mm |
| Shoots | | |
| shape | Erected, cross section: | Semi-erected, cross |
| | oblate, surface: with | section: circular, sur- |
| | stripes, nodes and in- | face: smooth, nodes |
| | ternodes without hairs | and internodes without |
| | | hairs |
| color | internodes with red | Internodes with green |
| | and green pigmenta- | color on ventral and |
| | tion on both sides, | dorsal side, nodes with |
| | nodes with red and | green ventral and |
| | green dorsal side and | dorsal side |
| | green ventral side | |
| Tendrils | | |
| form | Bifid or trifid | bifid |
| color | Green | Green |
| texture | normal | normal |
| number | 2 or <2 consecutive | 2 or <2 consecutive |
| Buds | | |
| size | average | average |
| shape | round | round |
| color | Light brown | Light brown |
| number | 2/node | 2/node |
| time of budbreak | early | early-medium |

LEAVES

| | | |
|---|---|---|
| Size | small-medium | medium |
| Number of leaflets | 5 | 5 |
| Glossiness | Medium-high | medium |
| Cross section shape | V-shaped | V-shaped |
| Color (immature) | | |
| Upper surface | Light green | Light green |
| Under surface | green | green |
| Color (mature) | | |
| Upper surface | Medium green | Medium green |
| Under surface | Darker green | Darker green |
| Petiole | | |
| Length (cm) | 6.2 | 6.0 |
| Color (upper surface) | green | green |
| Color (under surface) | Sometimes reddish | green |
| Stipule orientation | n.a. | n.a. |

FLOWERS

| | | |
|---|---|---|
| Flowering period | End of May | End of May |
| (time of beginning of | | |
| flowering) | | |
| Sex | hermaphrodite | hermaphrodite |
| Size | Average | average |
| Diameter (cm) | 0.3 (stamens) | 0.3 (stamens) |
| Fragrance | average | average |
| Flower number (at | n.a. | n.a. |
| 3^{rd} node from tip of | | |
| lateral mean and | | |
| range) | | |
| Petals | | |
| Length (cm) | n.a. | n.a. |
| Width (cm) | n.a. | n.a. |
| Overall shape | calyptra | calyptra |
| Color (immature) | green | green |
| Upper surface | | |
| Under surface | | |
| Color (mature) | green | green |
| Upper surface | | |
| Under surface | | |
| Sepals | | |
| Length (cm) | n.a. | n.a. |
| Width (cm) | n.a. | n.a. |
| Overall shape | n.a. | n.a. |
| Color (immature) | n.a. | n.a. |
| Upper surface | | |
| Under surface | | |
| Color (mature) | n.a. | n.a. |
| Upper surface | | |
| Under surface | | |
| Pedicel | | |
| Length (mm) | 45 (cluster) | 70 |
| Color | green | green |

FRUIT

| | | |
|---|---|---|
| Primocane time of | August 25th | September 7th |
| fruiting (1^{st} pick) | | |
| Clusters | | |
| cluster weight | g 170 | g 230 |
| cluster shape | conical | conical |
| cluster length | mm 180 | medium |
| avg. berries per | 200 | 220 |
| cluster | | |
| avg. clusters per | 2 | 2 |
| shoot | | |
| Berries | | |
| Berry size | Very low to low (g 1.8) | Very low to low (g 1.5) |
| Berry length (cm) | 1.76 | 1.50 |
| Berry width (cm) | 1.56 | 1.50 |
| Overall shape of | broad ellipsoid | globose |
| berry | | |
| Berry Texture | soft | soft |
| Berry Color | green | green |
| (immature) | | |
| Berry Color (mature) | green | green |
| Soluble solids (%) | 22.6 | 21.1 |
| Titratable acidity (as | 4.6 | 5.7 |
| g/L tartaric acid) | | |
| Sugar/acid ratio | 4.9 | 3.7 |
| Firmness | soft | soft |
| Seeds | 2-3 | 2-3 |
| Skin cracking? | no | rarely |
| Juice color | transparent | transparent |
| Berry taste | Neutral | neutral |
| Eating quality | n.a. | n.a. |
| Berry uses | wine | wine |
| Shipping quality | n.a. | n.a. |

What is claimed is:

1. A new and distinct grapevine, referred to as ‘FLEURTAI’, as herein described and illustrated by the characteristics set forth above.
